(19) United States
(12) Patent Application Publication
(10) Pub. No.: US 2020/0009561 A1
(43) Pub. Date: Jan. 9, 2020

(54) TOOLS AND METHODS FOR ISOLATION AND ANALYSIS OF INDIVIDUAL COMPONENTS FROM A BIOLOGICAL SAMPLE

(71) Applicant: Spin Bio, LLC, San Diego, CA (US)

(72) Inventors: Daniel J. Solis, Escondido, CA (US); Edward H. Cho, San Diego, CA (US)

(21) Appl. No.: 16/572,067

(22) Filed: Sep. 16, 2019

Related U.S. Application Data
(62) Division of application No. 15/408,411, filed on Jan. 17, 2017.
(60) Provisional application No. 62/279,719, filed on Jan. 16, 2016.

(51) Int. Cl. B01L 3/00; G01N 1/30; C12Q 1/6806

(52) U.S. Cl. CPC B01L 3/502753; G01N 1/30; C12Q 1/6806; B01L 3/50273; B01L 2400/0409; B01L 2300/0819; B01L 2400/0457; B01L 2400/0406; B01L 2300/0861

(57) ABSTRACT

The present invention describes a device(s) and assay(s) for the isolation and analysis of individual components from a sample. The invention provides a means of both isolating a multitude of individual components into an organized array and the subsequent analysis of such components by various detection and analysis methodologies. The invention provides a significant advancement in both the number of individual components that can be individually analyzed as well as enabling the quality and number of analytical methodologies that can be applied to them.

### CROSS-REFERENCES TO RELATED APPLICATIONS

This application claims the benefit of U.S. Provisional Application No. 62/279,719, (EFS ID: 24644430) filed on Jan. 16, 2016, the entire disclosure of which is hereby incorporated herein by reference for all purposes.

### FEDERALLY SPONSORED RESEARCH

Not Applicable

### SEQUENCE LISTING OR PROGRAM

Not Applicable

### FIELD OF THE INVENTION

The present invention relates generally to the area of isolating single components from a biological sample to separate sub-components for biochemical analysis.

### BACKGROUND OF THE INVENTION

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The invention described is aimed at better understanding the heterogeneous nature of biological samples, however the power to individualize components of a biological sample and maintain their individuality through multiple processing and analysis steps will have significance for all biological samples and will enable multi-omic analysis of both Homo- and Heterogeneous samples.

Recent advances in biotechnology have begun to unravel the underlying mechanisms surrounding the molecular pathogenesis of human disease. However, fundamental biological questions remain surrounding the true origins for the pathogenesis of human diseases such as cancer (Aceto et al. 2014). Despite the recent advances in biotechnology, additional breakthroughs in the way we examine human disease are needed to truly understand disease origin and progression in individual patients. More recent advances in single cell analysis technologies have demonstrated that many diseases may fundamentally arise from the reaction of single cells to their environment (Kohane et al. 2015). Unfortunately, these questions remain extremely difficult to answer given the variability and limited material from clinical samples. While gaining a dynamic, cellular understanding of disease genesis and progression is the key to realizing the potential of personalized medicine, it will require significant advancements in the way in which clinical samples are screened and qualified prior to analysis, specifically sample throughput, multi-omic analysis, and total cost.

Current approaches to single cell analysis are limited in their application by laborious workflows (laser microdissection, flow cytometry), complex consumables (microfluidic, droplet based) and exponential sample costs. Laser microdissection and flow cytometry require significant efforts to obtain single cells, and generally obtain fewer than 340 cells per sample. Also, they do not currently multiplex downstream analysis and therefore require a complete reagent kit per cell.

Microfluidics has recently emerged as a lower cost alternative and has been making advances in increasing the number of cells/sample with some commercial systems that can process 800 cells up from 96. This represents less than 0.2% of even the smallest Fine Needle Aspirate Biopsy (FNAB) (500,000-5M cells, Rajer et al. 2005). Other commercial miniaturized microtitre plate technologies are limited to 5,184 wells/chip due to custom Pico-liter liquid handlers. Even for the most recent technologies that have debuted, the maximum number of single cells that can be captured and analyzed is <45,000 (Macosko et al. 2015).

Previous technologies using microfluidics to isolate single cells rely mainly upon laying out microfluidic channels in-plane with the substrate material. This dramatically reduces the scalability for a device to maintain a consistent footprint. Many of these technologies rely on conjugation of biological material to functionalized beads to collect biological sub-components of interest, such as nucleic acids.

To better understand disease etiology and heterogeneity and gain a more comprehensive understanding of the cellular nature of disease, the number of cells analyzed needs to be increased exponentially and the total cost of analysis to drop inversely. Precision and personalized medicine will ultimately be an issue of statistics; more cells that can be analyzed from a patient sample will give a higher probability of finding the correct molecularly targeted treatment options and strategies for health care providers.

Analyzing individual or small groups of components from biological samples, particularly those encapsulated in a biomolecular carrier, in an addressable manner has proven to be difficult for current technologies. There is thus a need for a device and method that enables rapid, cost-effective isolation of hundreds of thousands, to millions of individual components of a sample such that multiple downstream analyses can be performed. The present invention addresses this need with vertical microchannel arrays.

Although we will specifically discuss isolation of components from single cells as a specific example here, this invention applies to any biological sample either with components encapsulated in a capsule, components that can be encapsulated or components that can be bound to a physical entity such as a bead, microcapsule or vesicle capable of being captured, at least partially, based on physical size.

### SUMMARY OF THE INVENTION

The present invention relates generally to a high-capacity fluidic device with vertical fluidic channels used for isolating single components from a biological sample to separate sub-components for biochemical and molecular biological analysis. In some aspects of these methods, the biological sample is from animal or plant fluid biopsy samples. In some aspects of these methods, the biological sample is from animal or plant tissue samples. In certain embodiments, the invention provides methods for isolating single cells into an arrayed format that allows for addressable observation and analysis by being optically compatible with optical and fluorescence microscopes. In certain embodiments, the invention provides methods for isolating single cells into an array allows for addressable biochemical analysis physical and chemical separation and isolation of sub-components, such as nucleic acids, proteins, etc., from other sub-components. In certain embodiments, the invention provides methods for isolating single cells into an array for individual testing or treatment with chemical compounds by being able to deliver chemicals individually or in bulk to each well in the array.

In certain embodiments, the invention provides methods for analyzing biological molecules from a single cell. This can include, but is not limited to, nucleic acids for genotyping, proteins for phenotyping, virus or bacterial to measure microbial infections, etc.

In certain embodiments, the invention provides methods for analyzing biological molecules from a small biological sample.

In certain embodiments, the invention provides a through-substrate fluidic device for the isolation, processing and analysis of individual biological components from a sample. In certain embodiments, the invention is for a high-density array of vertical fluidic structures consisting of at least a capture well and vertical through-channel of identical or various sizes wherein individual components of a biological sample, such as individual cells, can be isolated via the application of a force, such as by centrifugation, capillary action, gravity, or pressure.

In certain embodiments, the device is a fluidic containing a sample inlet, reservoir, and outlet that allows for the application of a sample to a substrate containing an array of vertical, through the substrate, fluidic channels.

In certain embodiments, the size and aspect ratio of the invention can be configured and adjusted to mate with other physical assay devices or to customize for assays. This includes adjustments to size of array capture wells, pitch between array capture wells, and size of vertical fluidic channels. The vertical, through-substrate nature of the fluidics enables a high-density of channels per unit area, dramatically increasing the number of components that can be individualized, processed, and analyzed. This vertical channel form factor allows for exponential increases in the number of components that can be individualized, processed, and analyzed either by increasing size of the device, the density of the channels, or the design layout of the channels.

In certain embodiments, the configuration of the array can be altered and additional components such as gaskets can be added that allows for coupling of multiple devices or other attachable devices on the same chip to analyze multiple samples.

In one embodiment of the invention, the high density of features allows for the mating of printed microarrays to the substrate containing the through-substrate fluidics. The high density of features is equivalent to that found in microarray applications. This embodiment of the invention enables the application of biological molecules required for performing multi-plexed, coded assays on the device.

In a certain embodiment, the ability for the invention to process samples non-destructively also enables a multi-omic (genomic, proteomic, transcriptomic, metabolomic, etc.) analysis of individual components.

### BRIEF DESCRIPTION OF THE DRAWINGS

FIG. 1 illustrates the general layout of the multi-dimensional vertical channel array. The high feature density can be determined by the 200 micron scale bar, with the illustrated layout having over 180,000 individual vertical channels.

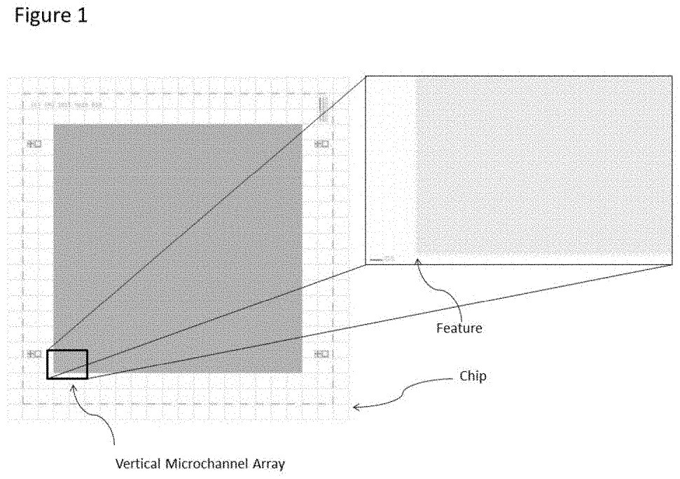

FIG. 2 illustrates the substrate and the vertically integrated capture well and channel. A 3D model illustrates the vertical, through substrate nature of the vertical channels.

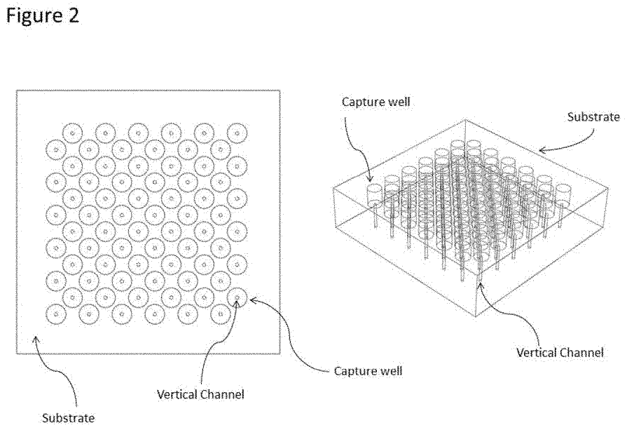

FIG. 3 illustrates a side-view of the device, showing the cross-section of the substrate with vertical channels.

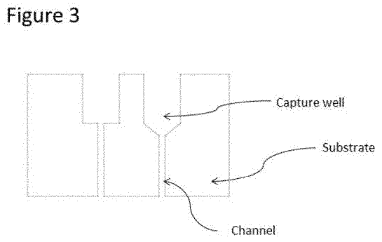

FIG. 4. illustrates a mode of operation in which the sample is applied to the device and placed under centrifugation to populate the individual features with analyte from the sample. Analyte remaining outside of the capture well can be flushed from the device prior to further processing. Additional reagents can be applied to the device and the device can be placed under a gradient, chemical, thermal or photonic (e.g., application of a light source) exposure.

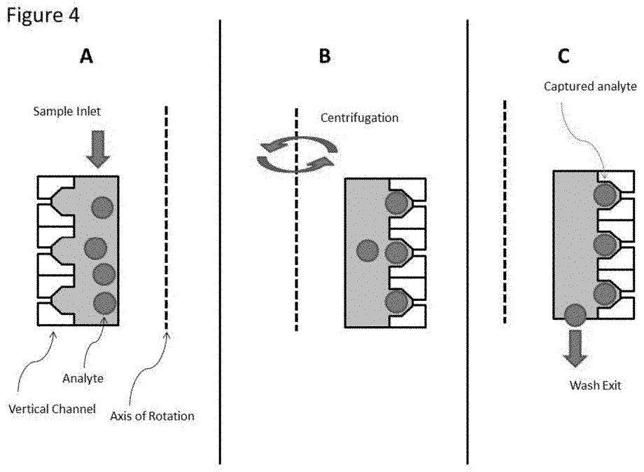

FIG. 5. illustrates the ability of the device to discriminate sample components based on physical attributes, including, but not limited to, physical size, Youngs modulus, or plasticity, for example.

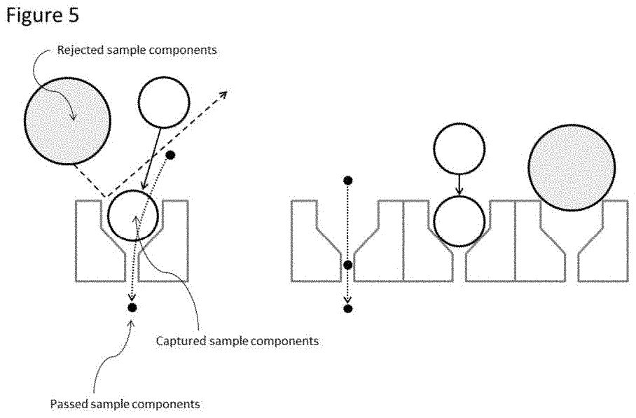

FIG. 6. illustrates the ability of the device to separate cargo from an analyte for further downstream processing by application of force, including, but not limited to, centrifugation, wherein the relationship between the analyte and cargo is maintained based on the addressable individuality of the vertical channels.

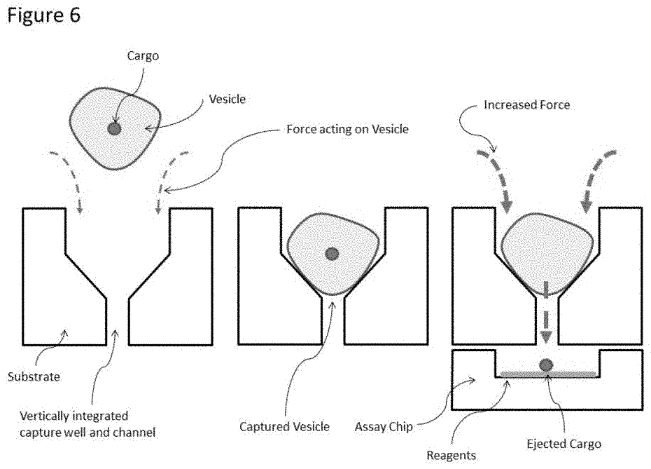

FIG. 7. illustrates the ability of the device to process multiple types of cargo within an analyte, wherein the cargo may be separated by use of applied force as illustrated in FIG. 6, or by the application of a gradient that disrupts the analyte causing release of the cargo. Said cargo can then be processed and or analyzed on an assay chip, wherein the assay chip can be either a separate substrate or the same substrate as the device containing the vertical channels.

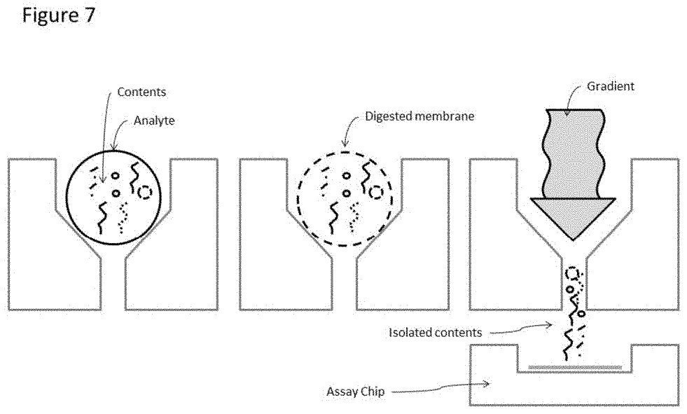

### DETAILED DESCRIPTION OF THE INVENTION

Note that, the singular forms “a,” “an,” and “the”, as used both within the application and in the appended claims, include plural referents. Thus, unless the context clearly dictates otherwise, reference to “a vertical fluidic channel” refers to one or more copies of a vertical fluidic channel, and reference to “the isolation of cells . . . ” includes reference to equivalent steps and methods known to those skilled in the art.

Unless defined otherwise, all technical and scientific terms used herein have the same meaning as commonly understood by one of ordinary skill in the art to which this invention belongs. All publications mentioned in the present description are referenced for describing and disclosing articles and methodologies that may be used in relation with the described invention.

Wherein a range of values is given, it is understood that the intervening values between the upper and lower limit of that range, inclusive of any other stated or intervening value in that stated range is encompassed within the invention.

In the following description, details are provided to enable a more thorough understanding of the invention that are not requisite in their entirety to enable the present invention, as should be evident to one skilled in the art. Also, features and procedures common to those skilled in the art have not been described.

### The Invention

The present invention provides a device and methods for isolation and analysis of individual components from a biological sample. The invention provides isolation of individual components from a sample by isolating them within a vertical fluidic channel. The vertical nature of the channel enables a higher density of channels to be fabricated within a given area. This ability to array the channels on a substrate both enables a high efficiency in isolation of components as well as provides a powerful means by which said components can be analyzed. The arrayed features enable visualization of the isolated components as well as the ability to introduce chemical entities capable of eliciting a detectable signal based upon interaction or interactions with said isolated component. The arrayed, high density vertical fluidic channels also enable the integration of unique chemical entities, capable of generating a detectable signal, that due to the uniqueness of the chemical entity, can be analyzed separate from the device, but traceable back to the individual channel and or isolated component.

### Components

Biological samples contain many types of components that represent various functions. The present invention describes the isolation of individual cells as the preferred embodiment but is not meant to be limiting as those skilled in the art will recognize that the mechanism of isolation is not enabled by the component being a cell, but rather by the physiochemical nature of the component. It will be also evident to those skilled in the art that there exists methods to vary the effective physiochemical nature of an entity that does not permanently alter is biological function. For example, encapsulation of cells within a droplet of uniform size would enable one to capture a larger range of cell sizes and cell types than direct capture. Another example would be the reversible binding of an entity of interest onto a bead such that the entity could be isolated by the physical size of the bead, rather than the entity of interest.

### Vertical Fluidic Channel

A vertical fluidic channel is defined as being formed through the substrate, as opposed to along the substrate, and having a multitude of dimensions along the long axis of the channel and being definable in its location on said substrate. This feature of a multitude of dimension enables greater functionality than that of a filter type device or a device fabricated by random processes. The vertical fluidic channel, by nature of its predeterminable fabrication, can be arrayed in a multitude of configurations in which each channel is physical addressable in a given coordinate system, such as a Cartesian coordinate system defining both the x-axis and y-axis position of said vertical fluidic channels.

### Substrate

A substrate in this context is any material that can be processed to create a vertical fluidic channel through said substrate in a controlled and predetermined manner. The substrate also enable the introduction to the vertical fluidic channel or array of vertical fluidic channels, of the biological sample containing the individual component. The substrate also enable the incorporation of additional substrates containing chemical entities that may be used to elicit a detectable signal from the individual component isolated within the vertical fluidic channel. For example, silicon provides a substrate in which standard microfabrication processes and methods enable one skilled in the art to form vertical channels through the substrate, in predefined positions, with varying dimensions along said channel. For example the Bosch etch process is well known to be capable of forming vias through a silicon substrate, and that by varying said process the physical dimensions of said via can be controlled. By repeating the process with subsequent lithographic patterning steps and from both the top and bottom side of said substrate, a multitude of dimensions, with varying profiles can be achieved.

### Capture Surface

A capture surface is defined as being a substrate to which either the individual component of interest, or a sub-component which comprises a portion of said individual component has, through the method of utilization of the device, has a physical and or chemical interaction with during the course of operation of the device. For example, a microarray comprising an array of unique sequences of nucleic acid, may be incorporated into the device comprising the vertical fluidic channels, such that individual components isolated in said vertical fluidic channels may be processed by the introduction of chemical reagents to release nucleic material of which, in part, the individual component is comprised of and the interaction of said released nucleic acid material to the complimentary nucleic acid material on the capture surface such that a subsequent detectable signal can be generated by the enzymatic extension of the nucleic material that can be analyzed by sequencing technologies, such as next generation sequencing.

### Chemical Entity

A chemical entity herein describes any molecule or molecules that can generate a detectable signal based upon the presence or absence of either said chemical entity or through interaction with an additional chemical entity. For example, a molecule, such as a fluorophore, that in the presence of an interacting chemical entity, changes its ability to fluoresce would constitute a chemical entity capable of generating a detectable signal based upon the presence of said interacting molecule.

### REFERENCES

### U.S. Patent Documents

1. U.S. Pat. No. 5,837,200 November 1998 Diessel et al.
2. US20150051098A1 Feb. 2015 Chen et al.
3. U.S. Pat. No. 6,767,706 B2 Jul. 2004 Quake et al.
4. US 2005/0053952 A1 Mar. 2005 Hong et al.
5. U.S. Pat. No. 6,027,873 February 2000 Schellenberger et al.
6. U.S. Pat. No. 8,309,035 B2 Nov. 2012 Chen et al.
7. U.S. Pat. No. 6,338,802B1 Oct. 1998 Bodner et al.
8. U.S. Pat. No. 5,506,141 April 1996 Weinreb et al.

### Other Publications

1. Nicola Aceto et al., “Circulating Tumor Cell Clusters Are Oligoclonal Precursors of Breast Cancer Metastasis.,” Cell 158, no. 5 (Aug. 28, 2014): 1110-22, doi:10.10161/j.cell.2014.07.013.
2. Isaac S Kohane, “Ten Things We Have to Do to Achieve Precision Medicine.,” Science (New York, N.Y.) 349, no. 6243 (Jul. 3, 2015): 37-38, doi:10.1126/science.aab1328.
3. Evan Z Macosko et al., “Highly Parallel Genome-Wide Expression Profiling of Individual Cells Using Nanoliter Droplets.,” Cell 161, no. 5 (May 21, 2015): 1202-14, doi:10.1016/j.cell.2015.05.002.
4. Mirjana Rajer and Marko Kmet, “Quantitative Analysis of Fine Needle Aspiration Biopsy Samples,” Radiology and Oncology 39, no. 4 (2005): 269-72.

1-20. (canceled)

21. A method for isolating and analyzing individual cells or vesicles, comprising:
placing cells onto a substrate having a plurality of capture wells, a top, a bottom, and vertical fluidic channels that are formed through the substrate and below each capture well to provide a fluidic connection from the capture wells through the bottom of the substrate, wherein the capture wells are each configured for the isolation of a single cell or vesicle based on size, wherein the channels have definable locations on said substrate, and wherein an assay chip comprises a printed microarray and is attached below the substrate;
isolating individual cells or vesicles in the capture wells by centrifugation;
capturing nucleic acid contents from the individual cells or vesicles at addressable locations on the assay chip.

22. The method of claim 21, wherein the substrate comprises over 180,000 capture wells and vertical fluidic channels.

23. The method of claim 21, wherein the capture wells have a larger diameter on one face of the substrate and a smaller diameter on the opposite face.

24. The method of claim 21, wherein the capture wells have a multitude of dimensions no larger than about 500 microns and no smaller than 100 nm.

25. The method of claim 21, wherein the substrate comprises silicon, fused silica, glass, polycarbonate, acrylic, PDMS, polyethylene, silicon nitride, polyimide, or polystyrine, polyethylene terephthalate, polyetherketone, polyamide, polyoxymethylene, or polysulphone.

26. The method of claim 21, further comprising analyzing the individual cells or vesicles by optical or fluorescence microscopy.

27. The method of claim 21, further comprising placing the device under a gradient, chemical, thermal or photonic exposure to release the nucleic acid contents from the individual cells or vesicles.

28. The method of claim 21, further comprising centrifuging the substrate a second time with an increased force compared to the first centrifugation to eject the nucleic acid contents from the individual cells or vesicles.

29. The method of claim 21, further comprising applying a chemical reagent to the individual cells or vesicles.

30. The method of claim 21, further comprising detaching the assay chip from the substrate.

31. The method of claim 21, further comprising analyzing the nucleic acid contents by sequencing.

32. A method for isolating and analyzing individual cells or vesicles, comprising:
placing cells onto a substrate having a plurality of capture wells, a top, a bottom, and vertical fluidic channels that are formed through the substrate and below each capture well to provide a fluidic connection from the capture wells through the bottom of the substrate, wherein the capture wells are each configured for the isolation of a single cell or vesicle based on size, and wherein the channels have definable locations on said substrate;
isolating individual cells or vesicles in the capture wells by centrifugation;
attaching an assay chip comprising a printed microarray below the substrate;
capturing nucleic acid contents from the individual cells or vesicles at addressable locations on the assay chip; and
detaching the assay chip from the substrate.

33. The method of claim 32, wherein the substrate comprises over 180,000 capture wells and vertical fluidic channels.

34. The method of claim 32, wherein the capture wells have a larger diameter on one face of the substrate and a smaller diameter on the opposite face.

35. The method of claim 32, wherein the capture wells have a multitude of dimensions no larger than about 500 microns and no smaller than 100 nm.

36. The method of claim 32, further comprising analyzing the individual cells or vesicles by optical or fluorescence microscopy.

37. The method of claim 32, further comprising placing the device under a gradient to release the nucleic acid contents from the individual cells or vesicles.

38. The method of claim 32, further comprising centrifuging the substrate a second time with an increased force compared to the first centrifugation to eject the nucleic acid contents from the individual cells or vesicles.

38. The method of claim 32, further comprising applying a chemical reagent to the individual cells or vesicles.

40. The method of claim 32, further comprising analyzing the nucleic acid contents by sequencing.
